(19) United States
(12) Patent Application Publication
(10) Pub. No.: US 2006/0067972 A1
(43) Pub. Date: Mar. 30, 2006

(54) Devices for renal-based heart failure treatment

(75) Inventors: Randy J. Kesten, Mountain View, CA (US); Sophia Pesotchinsky, Los Altos Hills, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: FlowMedica, Inc., Fremont, CA (US)

(21) Appl. No.: 11/167,056

(22) Filed: Jun. 23, 2005

Related U.S. Application Data
(60) Provisional application No. 60/583,139, filed on Jun. 23, 2004.

(51) Int. Cl. A61K 38/18; A61K 31/7076; A61K 31/522; A61F 2/00

(52) U.S. Cl. 424/426; 514/2; 514/263.32; 514/308; 514/47; 424/608

(57) ABSTRACT

Methods and devices are provided to preferentially deliver biologically active substances to a patient's renal artery, particularly for the treatment of congestive heart failure. The apparatus will comprise an implantable depot which may be in the form of a biodegradable vascular implant, a replenishable reservoir, a drug pump, or combinations thereof. The reservoirs will preferentially be refillable using needles or other transcutaneous techniques.

### CROSS-REFERENCES TO RELATED APPLICATIONS

The present application is a non-provisional of U.S. Patent Application Ser. No. 60/581,139 (Attorney Docket No. 022352-002300US), filed Jun. 23, 2004, the full disclosure of which is incorporated herein by reference.

### BACKGROUND OF THE INVENTION

1. Field of the Invention

This invention pertains generally to devices for renal-based heart failure treatment, and more particularly to devices that increase renal perfusion and thereby remove excess fluid and improve cardiac function of CHF patients.

The proper function of the kidney is directly related to cardiac output and related blood pressure into the renal system. These physiological parameters, as in the case of congestive heart failure (CHF), may be substantially compromised. Thus, patients suffering from CHF would receive enormous benefit from safe and effective modalities for local therapies or prophylaxis of renal conditions or compromised function as such relates to CHF. Such renal function in CHF patients may also be further compromised during a surgical intervention such as an angioplasty, coronary artery bypass, valve repair or replacement, or other cardiac interventional procedure. A patient undergoing these procedures may be particularly susceptible to renal damage from contrast imaging.

In conventional therapies, patients presenting emergently with pulmonary edema and related symptoms related to CHF are often placed on diuretics or vasodilators systemically in the hopes of increasing kidney function and thus reduce edema. However, because of already low cardiac output and blood pressure, these systemically administered agents do not often find their target in the kidneys in substantial concentration, and as such may take a relatively long time to achieve any beneficial result. In the meantime, systemic side effects (i.e., hypotension) of such agents often force their discontinuation prior to reaching their desired effect.

Therefore, there exists a real clinical need for a means to achieve significant fluid overload reduction quickly, reliably, and in the absence of untoward side effects in the CHF patient population that often presents with pulmonary edema in the presence of many other serious co-morbidities.

In the particular case of CHF, though also related to other conditions and interventional situations, relatively long dwell times may be desired for prolonged local administration of renal protective agents into the renal system. While such may be achieved via retrograde femoral approach, an antegrade approach such as via the brachial arteries may be desired in many circumstances (in particular for relatively long dwell times where a patient may not be required to lay relatively flat). However, patient motion during such dwell periods may be dislodging to conventional devices. This includes arm and upper body motions for example in a brachial approach, or leg and waist or lower body motion in a femoral approach to the renal arteries.

Therefore, a need also exists for a bilateral renal delivery device system and method that allows for continuous and robust positioning of the bilateral delivery/injection assembly in-vivo despite relative motion between the intravascular access site and the injection site.

It is further appreciated that, each year, large numbers of patients are exposed to contrast media associated with diagnostic imaging and treatment procedures. Various diagnostic systems and procedures have been developed using local delivery of dye (e.g. radiopaque “contrast” agent) or other diagnostic agents, that allow an external monitoring system to gather important physiological information based upon the diagnostic agent's movement or assimilation in the body at the location of delivery and/or at other locations affected by the delivery site. Angiography is one such practice using a hollow, tubular angiography catheter for locally injecting radiopaque dye into a blood chamber or vessel, such as for example coronary arteries in the case of coronary angiography, or in a ventricle in the case of cardiac ventriculography.

Other systems and methods have been disclosed for locally delivering a therapeutic agent into a particular body tissue within a patient via a body lumen. For example, angiographic catheters of the type just described above, and other similar tubular delivery catheters, have also been disclosed for use in locally injecting treatment agents through their delivery lumens into such body spaces within the body. More detailed examples of this type include local delivery of thrombolytic drugs such as TPA™, heparin, cumadin, or urokinase into areas of existing clot or thrombogenic implants or vascular injury. In addition, various balloon catheter systems have also been disclosed for local administration of therapeutic agents into target body lumens or spaces, and in particular associated with blood vessels. More specific previously disclosed of this type include balloons with porous or perforated walls that elute drug agents through the balloon wall and into surrounding tissue such as blood vessel walls.

Yet further examples for localized delivery of therapeutic agents include various multiple balloon catheters that have spaced balloons that are inflated to engage a lumen or vessel wall in order to isolate the intermediate catheter region from in-flow or out-flow across the balloons. According to these examples, a fluid agent delivery system is often coupled to this intermediate region in order to fill the region with agent such as a drug that provides an intended effect at the isolated region between the balloons.

The diagnosis or treatment of many different types of medical conditions associated with various different systems, organs, and tissues, may also benefit from the ability to locally deliver fluids or agents in a controlled manner. In particular, various conditions related to the renal system would benefit a great deal from an ability to locally deliver therapeutic, prophylactic, or diagnostic agents into the renal arteries.

Acute renal failure (“ARF”) is an abrupt decrease in the kidney's ability to excrete waste from a patient's blood. This change in kidney function may be attributable to many causes. A traumatic event, such as hemorrhage, gastrointestinal fluid loss, or renal fluid loss without proper fluid replacement may cause the patient to go into ARF. Patients may also become vulnerable to ARF after receiving anesthesia, surgery, or a-adrenergic agonists because of related systemic or renal vasoconstriction. Additionally, systemic vasodilation caused by anaphylaxis, and anti-hypertensive drugs, sepsis or drug overdose may also cause ARF because the body's natural defense is to shut down, i.e., vasoconstriction of non-essential organs such as the kidneys. Reduced cardiac output caused by cardiogenic shock, congestive heart failure, pericardial tamponade or massive pulmonary embolism creates an excess of fluid in the body, which can exacerbate congestive heart failure. For example, a reduction in blood flow and blood pressure in the kidneys due to reduced cardiac output can in turn result in the retention of excess fluid in the patient's body, leading, for example, to pulmonary and systemic edema.

The renal system in many patients may also suffer from a particular fragility, or otherwise general exposure, to potentially harmful effects of other medical device interventions. For example, the kidneys as one of the body's main blood filtering tools may suffer damage from exposure to high-density radiopaque contrast dye, such as during coronary, cardiac, or neuroangiography procedures, of the type mentioned above. One particularly harmful condition known as “radiocontrast nephropathy” or “RCN” is often observed during such procedures, wherein an acute impairment of renal function follows exposure to such radiographic contrast materials, typically resulting in a rise in serum creatinine levels of more than 25% above baseline, or an absolute rise of 0.5 mg/dl within 48 hours. Therefore, in addition to congestive heart failure (CHF), renal damage associated with RCN is also a frequently observed cause of ARF. Radiocontrast induced nephropathy is one of the most common causes of hospital onset renal failure and renal impairment in hospital patients. While most patients recover the majority of renal function, a minority become dialysis dependant. Accordingly, such conditions are further situations where improved local delivery devices and procedures may provide substantial benefit to patient healthcare.

2. Description of the Background Art

Catheters and systems for delivering blood and other agents to the renal arteries are described in U.S. Pat. No. 6,749,598 and published U.S. Application Nos. 2004/0064091; 2004/0064090; 2004/0064089; 2002/0173742; and 2002/0169413.

### BRIEF SUMMARY OF THE INVENTION

The present invention provides drug delivery devices and articles as well as methods for delivering drugs to a patient's renal arteries, particularly for the treatment of congestive heart failure. The apparatus of the present invention comprises implantable depots for releasing the biologically active substance locally into the patient's renal arteries. In the first instance, the depot may comprise a biodegradable vascular implant, such as a stent, graft, scaffold, or other structure which can be implanted in the blood vessel, typically the aorta or the renal artery, and which can release the biologically active substance at a controlled rate for a predetermined time period. Such biodegradable vascular implants may be composed of natural polymers, such as collagen, elastin, hyaluronic acid, or other biological polymers which can be formed into a structure capable of being implanted in the blood vessel, typically a tubular structure which will be implanted on the wall of the blood vessel and provide a central lumen for blood flow therethrough. The depot structure will degrade over time, releasing the drug into the blood flow and ultimately into the renal arteries.

Alternatively, the biodegradable vascular implants may be formed from biodegradable synthetic polymers, such as polylactic acids, polyglycolic acids, and the like. Such synthetic biodegradable polymers will also preferably be formed into tubular structures which incorporate the biologically active substance therein and release the substance overtime as they degrade within the vascular environment.

Additionally, the implantable depot may comprise a reservoir having an open volume for holding and releasing the biologically active substance. Preferably, reservoirs will be refillable so that, after their initial charge is depleted, they may be refilled with the same or a different biologically active substance.

In a preferred example, the reservoirs will be transcutaneously accessible to permit replenishment with biologically active substance. For example, the reservoirs may have needle-penetrable septums that allow filling of the reservoir using a syringe in a conventional manner.

Still further optionally, the reservoirs may be connected to implantable pumps for precisely controlling the amount and/or timing of delivery of the biologically active substance to the renal arteries, aorta, or other points within the vasculature intended to deliver the substances to the renal arteries. Such pumps will typically be battery powered, optionally using rechargeable batteries which can be externally recharged using radiofrequency chargers.

Examples of biologically active substances that may find use in the implantable depots and methods of the present invention include, without limitation: vasodilators, including for example papavarine, fenoldopam mesylate, calcium-channel blockers, acetylcholine, nifedipine, nitroglycerine, nitroprusside, adenosine, dopamine, and theophylline; antioxidants, such as for example acetylcysteine; diuretics, such as for example mannitol, and furosemide; and natriuretics, such as for example atrial natriuretic peptide (ANP), brain natriuretic peptide (BNP), C-type natriuretic peptide.

The present invention still further comprises methods for delivering such biological active substances to a patient's renal arteries, particularly when the patient suffers from or is at risk of suffering from congestive heart failure. A method comprises implanting a depot which, when implanted, can release the substance preferentially into at least one renal artery. The depot may be a biodegradable implant, may comprise a reservoir, may comprise a pump, and may allow for transcutaneous refilling, all that is generally described above in connection with the apparatus of the present invention. The methods may be adapted to deliver directly into one or more renal arteries, into the aorta above the renal arteries, or into other points in the vasculature which preferentially deliver the substances to the renal arteries. The preferred drugs to be delivered are also listed above.

An aspect of the invention is an implantable biodegradable stent that releases renal-specific compounds, such as vasodilators or diuretics.

Another aspect of the invention is a catheter-accessible renal-artery implant that contains a reservoir that can be refilled with such compounds and will gradually deliver them to the kidney.

A further aspect is the reservoir may be accessible through an implanted port in the patient's abdominal wall.

A still further aspect of the invention is an implantable pump, similar to insulin-infusion pumps, that is implanted such that its output flows into the renal artery, and that slowly infuses vasodilators or diuretics or other compounds directly to the kidneys. Optionally, sensors and controllers may be provided for controlling the pump in response to, for example blood pressure, renal blood flow, renal blood flow velocity (e.g., measured by Doppler ultrasound), electrolyte levels, hormone levels (e.g., BNP), comparative waste product concentrating in the renal arteries, and the like.

Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

### BRIEF DESCRIPTION OF THE DRAWINGS

FIGS. 1A and 1B illustrate alternative placements of biodegradable implants for releasing a biologically active substance to the renal arteries in accordance with the principles of the present invention.

FIGS. 2A and 2B illustrate alternative implantation sites for a drug reservoir for releasing biologically active substances to the renal arteries.

FIGS. 3A and 3B illustrate a combination of a drug reservoir and pump for delivering biologically active substances into the renal arteries according to the principles of the present invention.

FIGS. 4A and 4B illustrate use of port for replenishing a non-degradable vascular depot according to the methods of the present invention.

### DETAILED DESCRIPTION OF THE PRESENT INVENTION

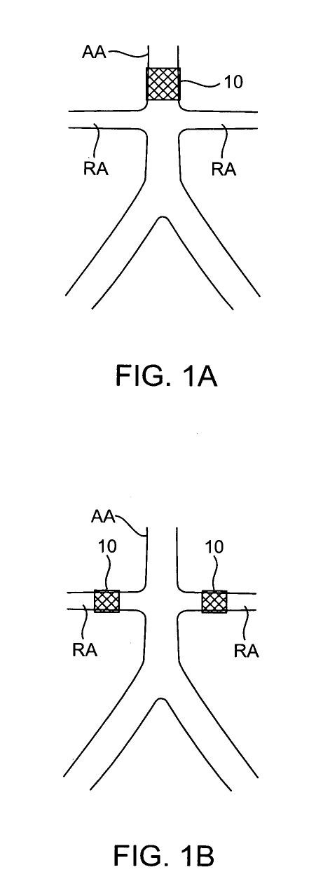

Referring to FIGS. 1A and 1B, biodegradable implants 10 carrying the bioactive substances of the present invention may be implanted at different regions in the vasculature to deliver the biologically active substance to the renal arteries RA. As shown in FIG. 1A, the biodegradable implant may be implanted in the abdominal aorta AA directly above the renal arteries. As the implant degrades over time, a biologically active substance which is incorporated in the implant will be released and will flow into the renal arteries. Alternatively, one or two biologically degradable implants 10 may be implanted directly within the renal arteries RA, as shown in FIG. 1B.

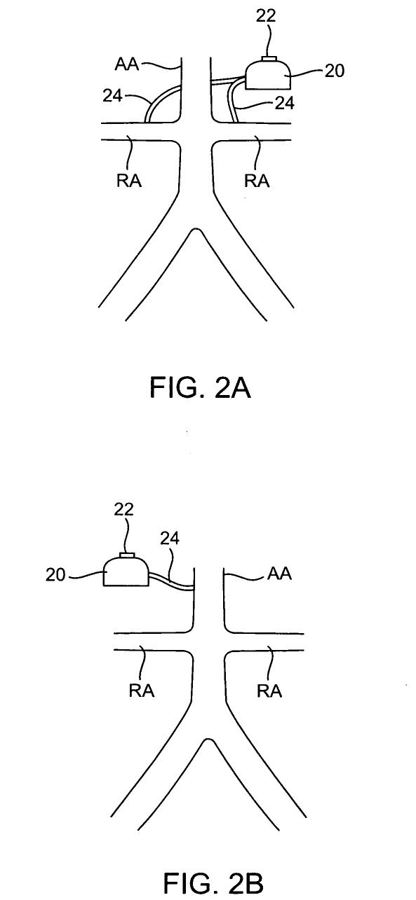

The present invention may also employ reservoirs having internal volumes for maintaining and releasing biologically active substances. As shown in FIGS. 2A and 2B, an implant 20 having a septum 22 may be implanted adjacent the renal arteries RA. In FIG. 2A, the reservoir 20 is connected via a pair of implanted tubes 24 to each of the renal arteries. In FIG. 2B, a single tube 24 is connected to the abdominal aorta above the renal arteries RA. The reservoirs 20 may be replenished from time to time with the biologically active substance by accessing the reservoirs via the septum port 22 using a relatively small gauge needle which can deliver the substance without coring the septum.

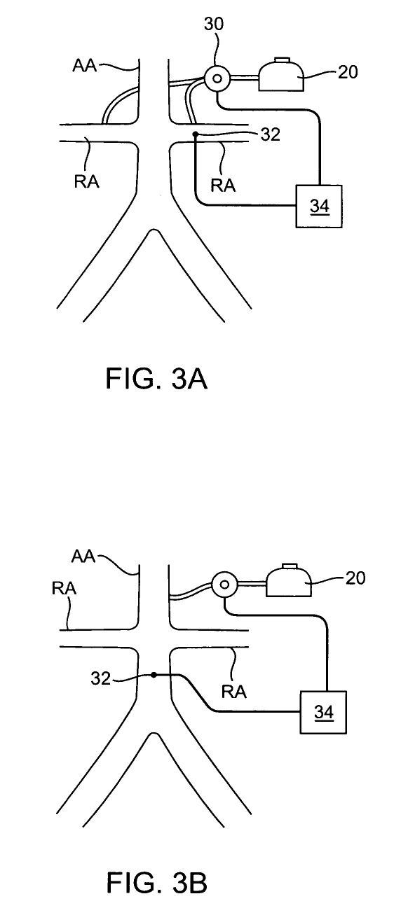

The reservoirs 20 may be combined with a powered, implantable pump 30 to enhance and control delivery of the substance from the reservoir to the renal arteries RA (FIG. 3A) or the abdominal aorta AA (FIG. 3B). Optionally, sensors 32 and controllers 34 may be provided to permit control of the amount of substance delivered by controlling the pump 30. The sensors may be implanted at various locations in the vasculature or elsewhere and may measure a number of patient conditions, such as blood pressure, renal blood flow, renal blood flow velocity, electrolyte levels, hormone levels, and comparative waste product concentrations. Based on these measurements, control circuitry in the controller 34 can titrate the delivery of biologically active substance from the pump to the patient. Such feedback control can rely on known control algorithms including proportional, derivative, and proportional-derivative control (PID), or could utilize special algorithms.

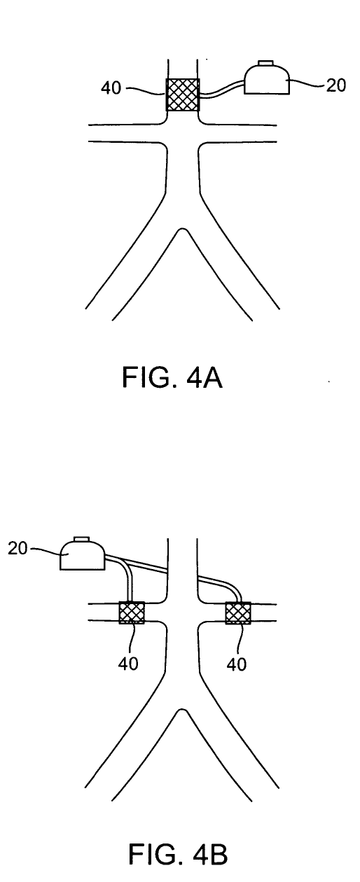

In a further alternative embodiment, the reservoirs 20 may be combined with replenishable depots 40 which are implanted within the abdominal aorta AA (FIG. 4A) or the renal arteries RA (FIG. 4B). The replenishable depots 40 will typically not be biodegradable and will contain pore structures which permit refilling with the biologically active substance and controlled release of the substance from the depots over time.

While the above is a complete description of the preferred embodiments of the invention, various alternatives, modifications, and equivalents may be used. Therefore, the above description should not be taken as limiting the scope of the invention which is defined by the appended claims.

What is claimed is:

1. An implantable depot for releasing a biologically active substance locally into the renal arteries.

2. An implantable depot as in claim 1, further comprising a biodegradable vascular implant which releases the biologically active substance, wherein the implant is implantable within the aorta near the renal arteries or within at least one renal artery.

3. An implantable depot as in claim 1, further comprising a reservoir for holding the biologically active substance.

4. An implantable depot as in claim 3, further comprising a pump connected to the reservoir for delivering the biologically active substance from the reservoir to the renal arteries.

5. An implantable depot as in claim 3, further comprising a port which is transcutaneously accessible to replenish the reservoir with biologically active substance.

6. An implantable depot as in any one of claims 1 to 5, wherein the biologically active substance is selected from the group consisting of vasodilators, antioxidants, and diuretics.

7. An implantable depot as in claim 6, wherein the biologically active substance comprises a vasodilator selected from the group consisting of papavarine, fenoldopam mesylate, calcium-channel blockers, acetylcholine, nifedipine, nitroglycerine, nitroprusside, adenosine, dopamine, and theophylline.

8. An implantable depot as in claim 6, wherein the biologically active substance comprises an antioxidant selected from the group consisting of acetylcystein.

9. An implantable depot as in claim 6, wherein the biologically active substance comprises a diuretic selected from the group consisting of mannitol and furosemide.

10. An implantable depot as in claim 6, wherein the biologically active substance comprises a natriuretic selected from the group consisting of atrial natiuretic peptide (ANP), brain natriuretic peptide (BNP), and C-type natgriuretic peptide.

11. A method for delivering biologically active substance to a patient's renal arteries, said method comprising:
implanting a depot which, when implanted, can release the substance preferentially into at least one renal artery.

12. A method as in claim 11, wherein the implant is a biodegradable implant which is implanted in the vasculature proximate the target renal artery(ies).

13. A method as in claim 12, wherein the implant is implanted in the aorta above the renal arteries.

14. A method as in claim 12, wherein the implant is implanted in at least one renal artery.

15. A method as in claim 11, wherein the implant comprises a refillable reservoir.

16. A method as in claim 15, further comprising periodically refilling the reservoir via a transcutaneous access route.

17. A method as in claim 15, further comprising pumping the biologically active substance from the reservoir to at least one renal artery.

18. A method as in any one of claims 11 to 17, wherein the biologically active substance is selected from the group consisting of vasodilators, antioxidants, and diuretics.

19. A method as in claim 18, wherein the biologically active substance comprises a vasodilator selected from the group consisting of papavarine, fenoldopam mesylate, calcium-channel blockers, acetylcholine, nifedipine, nitroglycerine, nitroprusside, adenosine dopamine, and theophylline.

20. A method as in claim 19, wherein the biologically active substance comprises an antioxidant selected from the group consisting of acetylcystein.

21. A method as in claim 19, wherein the biologically active substance comprises a diuretic selected from the group consisting of mannitol and furosemide.

22. A method as in claim 19, wherein the biologically active substance comprises a natriuretic selected from the group consisting of atrial natiuretic peptide (ANP), brain natriuretic peptide (BNP), C-type natriuretic peptide.
